(19) United States
(12) Patent Application Publication
(10) Pub. No.: US 2020/0294639 A1
(43) Pub. Date: Sep. 17, 2020

(54) AUTOMATED ADAPTATION OF PATIENT ASSESSMENT REPORT FORMAT

(71) Applicant: CURANTIS SOLUTIONS, Addison, TX (US)

(72) Inventors: Robert Parker FREEBURG, II, Addison, TX (US); Rishi Suresh KESHAV, Addison, TX (US); Mohsin NISAR, Addison, TX (US)

(21) Appl. No.: 16/352,720

(22) Filed: Mar. 13, 2019

(51) Int. Cl. G16H 15/00; G06F 17/24; G16H 10/60; G16H 30/40

(52) U.S. Cl. CPC G16H 15/00; G16H 30/40; G16H 10/60; G06F 17/248

(57) ABSTRACT

A method of generating a patient assessment report. The method comprises storing an assessment template in a data store, receiving values of a completed section of a patient assessment, and storing the values of the completed section of the patient assessment along with an associated section portion of the assessment template by an assessment component executing on a computer in the data store. The method further comprises reading a plurality of completed patient assessment sections by the assessment component from the data store and storing a completed patient assessment entry in an assessment table of the data store by the assessment component. The method further comprises reading the completed patient assessment entry from the data store by a report generation component executing on the computer and generating an assessment report based on the values of the patent assessment entry and based on the assessment template by the report generation component.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Patient care near the end of life may be partitioned into palliative care and hospice care. Hospice care is typically involved during the last six months of life or even just the last month of life. Hospice care is not typically directed to curing or restoring the health of the care patient but rather assuring their dignity and comfort as they approach death and possibly also assisting family and friends cope with the approaching death of the care patient. Palliative care may be involved over a longer period of time and precedes hospice care. Palliative care may be involved with helping patients maintain a good quality of life while in a slow decline caused by a chronic medical condition. Curative care, by contrast with both hospice care and palliative care, is directed to curing and/or restoring the care patient to a fully-functional health.

### SUMMARY

In an embodiment, a method of using three-tier architecture to dynamically generate a patient assessment report automatically adapted to an assessment format used in conducting a patient assessment is disclosed. The method comprises storing a first assessment template in a data store, wherein the first assessment template defines a presentation of a first patient assessment form by a patient care giver tool UI, receiving values of a completed section of a first patient assessment from the patient care giver tool UI by an assessment component executing on a computer system, and storing the values of the completed section of the first patient assessment along with an associated section portion of the first assessment template by the assessment component in an entry associated with the completed section of the first patient assessment in an assessment section data table of the data store. The method further comprises reading a plurality of entries associated with the completed sections of the first patient assessment by the assessment component from the assessment section data table and storing a first completed patient assessment entry in an assessment table of the data store by the assessment component, wherein the first entry comprises the values of the completed sections of the first patient assessment and the first assessment template. The method further comprises reading the first completed patient assessment entry from the assessment table from the data store by a report generation component executing on the computer system, where the report generation component comprises a report generation tool and a report generation utility library and providing the values of the completed sections of the first patient assessment and providing the first assessment template by the report generation tool to the report generation utility library. The method further comprises creating a first plurality of Java objects comprising the values of the completed sections of the first patient assessment by the report generation utility library based on the first assessment template and providing the first plurality of Java objects by the report generation utility library to the report generation tool. The method further comprises generating a first assessment report by the report generation tool based on the first plurality of Java objects, wherein the first assessment report presents the values of the first patient assessment in a first format based on the first assessment template. The method further comprises storing a second assessment template in the data store, wherein the second assessment template defines a presentation of a second patient assessment form by the patient care giver tool UI, receiving values of a completed section of a second patient assessment from the patient care giver tool UI by the assessment component, and storing the values of the completed section of the second patient assessment along with an associated section portion of the second assessment template by the assessment component in an entry associated with the completed section of the second patient assessment in the assessment section data table. The method further comprises reading a plurality of entries associated with completed sections of the second patient assessment by the assessment component from the assessment section data table and storing a second completed patient assessment entry in the assessment table of the data store, wherein the second entry comprises the values of the completed sections of the second patient assessment and the second assessment template. The method further comprises reading the second completed patient assessment entry from the assessment table from the data store by the report generation component and providing the values of the completed sections of the second patient assessment and providing the second assessment template by the report generation tool to the report generation utility library. The method further comprises creating a second plurality of Java objects comprising the values of the completed sections of the second patient assessment by the report generation utility library based on the second assessment template and providing the second plurality of Java objects by the report generation utility library to the report generation tool. The method further comprises generating a second assessment report by the report generation tool based on the second plurality of Java objects, wherein the second assessment report presents the values of the second patient assessment in a second format based on the second assessment template.

In another embodiment, a system for dynamically generating a patient assessment report automatically adapted to an assessment format used in conducting a patient assessment is disclosed. The system comprises at least one processor, a data store, a non-transitory memory, an assessment component stored in the memory, and a report generation component stored in the memory, wherein the report generation component comprises a report generation tool and a report generation utility library. When executed by the at least one processor, the assessment component stores a first completed patient assessment entry in an assessment table of the data store, wherein the first entry comprises values of a first patient assessment and a first assessment template that identifies the format of a presentation of a first patient assessment form by a patient care giver tool UI and stores a second completed patient assessment entry in the assessment table of the data store, wherein the second entry comprises values of a second patient assessment and a second assessment template that identifies the format of a presentation of a second patient assessment form by the patient care giver tool UI and wherein the second assessment template is different from the first assessment template. When the report generation component is executed by the processor, the report generation tool reads the first completed patient assessment entry from the assessment table from the data store, the report generation tool provides the first completed assessment entry to the report generation utility library and the report generation utility library creates a first plurality of Java objects comprising the values of the first patient assessment based on the first assessment template. When the report generation component is executed by the processor, the report generation utility library provides the first plurality of Java objects to the report generation tool, and the report generation tool generates a first assessment report based on the first plurality of Java objects, wherein the first assessment report presents the values of the first patient assessment in a first format based on the first assessment template. When the report generation component is executed by the processor, the report generation tool reads the second completed patient assessment entry from the assessment table from the data store and the report generation tool provides the second completed assessment entry to the report generation utility library. When the report generation component is executed by the processor, the report generation utility library creates a second plurality of Java objects comprising the values of the second patient assessment based on the second assessment template, the report generation utility library provides the second plurality of Java objects to the report generation tool, and the report generation tool generates a second assessment report based on the second plurality of Java objects, wherein the second assessment report presents the values of the second patient assessment in a second format based on the second assessment template.

In yet another embodiment, a method of dynamically generating a patient assessment report automatically adapted to an assessment format used in conducting a patient assessment is disclosed. The method comprises storing an assessment template in a format comprising a plurality of human readable attribute-value pairs in a column of a table in a data store, wherein the assessment template defines a presentation of a patient assessment form by a patient care giver tool UI as a plurality of sections of a patient assessment, receiving values of a completed section of a patient assessment from the patient care giver tool UI by an assessment component executing on a computer system, and storing the values of the completed section of the patient assessment along with an associated section portion of the assessment template by the assessment component in an entry associated with the completed section of the patient assessment in an assessment section data table of the data store. The method further comprises reading a plurality of entries associated with the completed sections of the patient assessment by the assessment component from the assessment section data table and storing a completed patient assessment entry in an assessment table of the data store by the assessment component, wherein the entry comprises the values of the completed sections of the patient assessment and the assessment template. The method further comprises reading the completed patient assessment entry from the assessment table from the data store by a report generation component executing on the computer system, where the report generation component comprises a report generation tool and a report generation utility library and providing the values of the completed sections of the patient assessment and providing the assessment template by the report generation tool to the report generation utility library. The method further comprises creating a plurality of Java objects comprising the values of the completed sections of the patient assessment by the report generation utility library based on the assessment template, providing the plurality of Java objects by the report generation utility library to the report generation tool, and generating a assessment report by the report generation tool based on the plurality of Java objects, wherein the assessment report presents the values of the patient assessment in a format based on the assessment template.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a block diagram of a system according to an embodiment of the disclosure.

FIG. 2 is another block diagram of the system according to an embodiment, of the disclosure.

FIG. 3 is a block diagram of data store tables according to an embodiment of the disclosure.

FIG. 4 is a block diagram of a report generation component according to an embodiment of the disclosure.

FIG. 5A and FIG. 5B are a flow chart of a method according to an embodiment of the disclosure.

FIG. 6 is a flow chart of another method according to an embodiment of the disclosure.

FIG. 7A, FIG. 7B, and FIG. 7C are a flow chart of a method according to an embodiment of the disclosure.

FIG. 8A and FIG. 8B are a flow chart of a method according to an embodiment of the disclosure.

FIG. 9 is a block diagram of a computer system according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

Typical health care is diagnostic driven and involves a kind of linear process of “solve for this” followed by “solve for that” actions. Hospice care is different and is not diagnostic driven but rather is plan of care driven. In hospice care, the patients are not expected to improve and recover and are experiencing a distinct decline in health that ends in their death. When federally allocated funds are involved in the hospice care, evidence of patient decline is desirably identified and documented by an interdisciplinary group (IDG). The IDG is populated by persons performing different roles, for example a doctor role, a nurse role, a spiritual advisor role, a social worker role, and a bereavement counselor role. The IDG meets periodically, for example about every two weeks, and findings of assessments of a patient at different IDG meetings are expected to manifest a trend of declining health of the patient. If, however, a patient exhibits signs of increasing health, they may be removed from hospice care.

Most patient care software tools are designed for typical health care, and often those patient care software tools are maladapted for use with the special needs involved in providing hospice care. Additionally, often care givers may use a plurality of different patient care tools that do not interoperate with each other and do not automatically share data on the same patient. Because of this lack of interoperability, care givers have to spend extra time jumping from one tool to a second tool and from the second tool back to the first tool to complete their work, which is inefficient. Additionally, this lack of interoperability interferes with members of an IDG accessing up-to-date information on hospice patients and interferes with meeting government regulations pertaining to qualification of patients for hospice reimbursement by the government. The present disclosure teaches a plan of care centric patient care giver tool system that is designed for providing hospice care. The patient care giver tool system taught herein is an integrated system that provides care givers seamless access to different tool functionalities from the same tool interface, and where the different tool functionalities have access to the same pool of patient data. While the focus in this patent disclosure is on generation of reports based on assessments of patients that automatically adapt the format of the assessments, this benefit is integrated with a three-tiered patient care giver tool system architecture. To more clearly describe the report generation, the whole three-tiered patient care giver tool system is described herein.

The plan of care for each different patient is generated and maintained independently for each patient and reflects each patient's initial condition when admitted to hospice care and the history of the subsequent evolution of the patient's health. While much of the following is directed to examples involving hospice care, it is noted that the teachings herein may advantageously be adapted for use with palliative care, which also differs in similar ways from typical health care processes.

The present disclosure teaches a patient care giver tool system crafted for use in a hospice environment that dynamically constructs a patient assessment user interface for a patient care tool using a three-tiered architecture. The system is designed to efficiently and flexibly support hospice care activities. Hospice care may be funded by government funds and may be subject to tight government cost controls. Historically, the hospice care environment has been under-served by software tools specifically designed for the hospice care environment, possibly because it has been difficult to make a paying business out of hospice care, and without profits, hospice care organizations cannot fund development of the desired software tools. The patient care giver tool system taught herein proposes to solve this computing problem and this economic problem concurrently, through its efficiency and flexibility as described hereinafter.

To perform efficiently, the system is designed to support a plurality of independent hospice care organizations from a single multi-tenant execution platform, for example from a cloud computing environment. Additionally, the integrated nature of the system promotes efficiency of hospice care giver operations. The hospice care givers, using this system, need not move from a first tool to a second tool and back to the first tool to complete their work. They can complete their tasks using a single tool interface extended to their portable devices (e.g., a tablet computer) and work with an integrated data store containing all relevant data pertaining to their patients, and where the data is continuously up-to-date. For example, care giver inputs into an assessment of a hospice patient can immediately affect the plan of care of the hospice patient, and the same care giver may immediately see a new order or intervention in the patient's plan of care resulting from their input. In a different system that is not integrated, it is unlikely that an assessment input provided in an assessment tool would immediately percolate into a separate plan of care tool for example. Likewise, the assessment inputs can be accessed through the same system immediately by the members of the IDG in evaluating a patient's current status and to validate the patient's hospice care eligibility according to federal guidelines, because the system assessment microservice operates on the same data store as the IDG microservice operates on.

Flexibility is desirable, because different independent hospice care organizations are expected to demand different functionality in the patient care tool. Additionally, the needs of a hospice care organization are expected to change and drive differences in the functionality of the patient care tool as their processes evolve and government regulations change over time. To promote flexibility, the system is designed to define functional aspects that are expected to change using data definitions in JSON (JavaScript Object Notation) format rather than be defined in database schemas and hard coded in scripts or computer programs. Functional and structural aspects that are defined in JSON format can be revised much more quickly than database schemas, code, or scripts can be changed to implement similar revisions. The patient care tool is designed to flow this flexibility through a plurality of complementary functionalities, including an assessment microservice and an assessment reporting tool. For example, when changes are made to an assessment form by editing a JSON document defining the assessment format, this format change automatically flows with assessment input data to the assessment reporting tool, whereby the reports generated by the assessment reporting tool automatically adopt the new assessment format.

The patient care giver tool system taught herein supports a role-based team care giving approach, where different personnel fulfilling the same role may be substituted for each other to provide patient care efficiently and timely. This team care giving approach promotes efficiency in scheduling delivery of care to patients, for example in choosing efficient driving routes to reduce driving mileage expenses. The team care giving approach promotes efficiency in completing IDG meetings to evaluate patients, where if one IDG member is called away in the middle of the IDG meeting—as sometimes happens in the hospice context—another person having the same role can substitute and the IDG meeting can continue without interruption. The patient care giver tool system supports a circulation process from assessments of patients to updates to a plan of care for each patient to the IDG meetings by providing shared access by each process step to the same body of patient data. In addition to promoting efficiency, this circulation process supports accuracy and prompt adaptation of the patient care plan.

The system provides different patient care functionalities from different microservices or modules that execute in a middle tier of the system architecture. For example, an assessment microservice is engaged for performing, recording, and reporting patient assessments. Other microservices may comprise one or more of a plan of care microservice, an interdisciplinary group (IDG) microservice, a scheduling microservice, an alerts and notifications microservice, a data analytics microservice, a history and event records microservice, a patient referral microservice, and a billing microservice. In an embodiment, at least some of the microservices extend a representational state transfer (REST) application programming interface (API) to a user interface (UI) application that executes in a front-end tier of the system architecture. In some contexts, the REST APIs may be referred to as RESTful APIs. A RESTful API uses hypertext transport protocol (HTTP) requests to GET, PUT, POST, and DELETE data. RESTful API technology provides some advantages such as relying on less bandwidth than a more robust communication technology such as simple object access protocol (SOAP).

In an embodiment, the UI application requests an assessment user interface format from the assessment microservice, processes the assessment user interface format to generate an assessment user interface document suitable to a specific user equipment (UE), and sends the assessment user interface document to the UE. The UE renders the assessment user interface document on a display of the UE and receives user inputs to controls presented in the display. The UE returns the user inputs to the UI application, and the UI application returns the user inputs to the assessment microservice via the RESTful API. The assessment microservice then acts upon the received user inputs, for example storing assessment data and assessment template data in a data store tier or back-end tier of the system architecture. In an embodiment, user interfaces of other microservices may likewise be automatically generated and sent to the UE in a manner similar to that described above for the assessment user interface.

An assessment user interface format comprises one or more section formats, each section format comprises one or more card formats, and each card format comprises one or more field formats. Each different card format is associated with a card type, and different card formats may be associated with the same card format. In an embodiment, card types comprise a list card type, a delta list card type, a questions card type, a form card type. In an embodiment, other card types are defined. A list card type is a list of data elements. In an embodiment, a list card type may be a list of name, value pairs. A delta list card type is a list of data elements, where each data element comprises a current value and a previous value, for example a current weight and a previous weight, a current blood pressure and a previous blood pressure. In an embodiment, a questions card type is a list of questions asked associated with a corresponding answer. In an embodiment, the section formats, card formats, and field formats for a given hospice organization are defined in a JSON document that is stored in a JSON-B column of an assessment relational database in the data store tier of the system architecture. The JSON-B column promotes inclusion of unstructured data (the JSON document contents is deemed unstructured data in that there is no static structure defined for the data by a schema and the structure is rather indicated by the JSON document itself as human readable attribute-value pairs) within an otherwise structured data table entry. In some contexts, the JSON document contained in the JSON-B column may be referred to as a JSON blob.

Different hospice organizations may provide different assessment type definitions and different format definitions, whereby to customize the assessment screens presented on UEs of hospice care givers of each different hospice organization. This definition may be referred to as an assessment template. The definition of the format of a section may be referred to as a section template; the definition of the format of a card may be referred to as a card template; and the format of a field may be referred to as a field template. By defining section formats, card formats, and field formats in JSON documents in JSON-B columns of tables in the data store tier, the system supports changing assessment interfaces based on changing the JSON document (e.g., changing data, configuring data) rather than based on changing database schemas, code, or scripts. Changing database schemas, computer code, and programming scripts may involve more effort—development effort, configuration control effort, software testing effort—than the effort involved in modifying data. The different assessment types and formats of different hospice organizations may be managed on the basis of differentiating between interface requests from UEs that identity themselves as associated with a specific hospice organization.

When a care giver completes a section of an assessment of a patient and moves on to a different section, the completed section is stored by the assessment microservice in the data store in the data store tier. The assessment input data is stored and the format of the section template and field template likewise is stored in the data store in the data store tier. When the section data and format are stored, the assessment microservice launches a thread that retrieves the saved assessment section input data and another thread that retrieves associated section scripts from the database. Each script may evaluate an assessment data value against a pre-defined script criterion. If the script criterion evaluates true (e.g., the input assessment data value satisfies the criterion), an issue is created and stored by the script into a plan of care for the hospice patient undergoing assessment. This issue in the plan of care may then lead to an associated goal being attached to the plan of care and subsequently an intervention associated to the goal being attached to the plan of care and ultimately care delivered to the patient being adapted based on the intervention attached to the plan of care.

When an assessment is completed, the assessment microservice creates a completed assessment entry from each of the plurality of completed section rows stored in a section table in the data store and stores the completed assessment entry in a completed assessment table in the data store. The completed assessment entry comprises the input of each of the completed assessment sections as well as the format information (e.g., assessment template information) of each of the completed assessment sections. In an embodiment, when invoked manually, a report generation component reads a completed assessment entry from the completed assessment table and generates a report containing the assessment section input values formatted according to the format of the assessment sections. The report generation component comprises a report generation utility library and a third party report generation tool. The report generation tool may not interwork with or understand the formats and card types of the assessment formats. The report generation utility library is linked with and used by the report generation tool to mediate access to the assessment format portions of the completed assessment entries. Said in other words, the report generation tool may access the assessment values and generate the assessment report in a format adapted to the assessment format (e.g., as presented on the user interface to a care giver performing the assessment) using methods provided by the report generation utility library. The report generation tool provides values of the completed sections of the assessment to the report generation utility library. The report generation utility library creates a plurality of Java objects comprising the values of the completed sections of the assessment based on the assessment template. The report generation utility library provides the Java objects to the report generation tool. The report generation tool generates the report based on the Java objects, and by this process presents the values of the completed assessment in a report that is automatically adapted to the assessment format used in conducting the patient assessment: the presentation format of the report is adapted to the assessment format presented on the user interface to the care giver when conducting the patient assessment. When an assessment is completed using a different format, as for example when an assessment is completed by a different organization using the same patient care giver tool system, the assessment report generated based on that completed assessment is automatically adapted to that different assessment format used by the different organization in conducting the patient assessment.

The methods provided by the report generation utility library may transcode the formats in the assessment format into a format supported by the report generation tool. In an embodiment, the report generation utility library transcodes the assessment input values and assessment format to Java objects such as Java list objects or such as Java Beans which are compatible with the report generation tool. The report produced by the report generation component may be considered to be an electronic document that can be presented on a display, printed by a printer, or transmitted as an electronic file. In an embodiment the report may be generated in a portable document format (PDF). In an embodiment the report may be generated as a byte stream that is sent to a UE 102, for example a UE 102 used by a hospice care giver and/or used by a member of the IDG. In an embodiment, the report generation tool is invoked automatically on the event of creating the completed assessment entry in the assessment table in the data store.

In an embodiment, the assessment report generated by the report generation component comprises a plurality of images of pain locations with reference to body parts. For example, the assessment report may comprise an image of a left leg of a patient indicating one or more locations where the subject patient reported experiencing pain during the assessment. The pain location indications may comprise a level of pain indication, for example a level from 0 to 5, from 1 to 5, from 0 to 10, from 1 to 10, or some other range of pain values. The images of pain locations with reference to body parts may comprise one or more of a leg body part image, an arm body part image, a neck body part image, a back body part image, an abdomen body part image, a head body part image, or an upper chest body part image. The assessment report presents the values of the patient assessment ordered by sections as the sections are ordered in the assessment template. Additionally, the assessment report may identify assessment sections, assessment cards, and assessment fields with names or titles. The assessment report may present the values within sections arranged according to card layouts and field layouts as would be seen in an assessment section presentation on a care giver tablet or other mobile communication device.

It is observed that the report generation component automatically adapts the format of the assessment report it produces as the assessment format used to carry out patient assessments changes, for example as a hospice care organization modifies and evolves the assessment template it uses to assess its patients. It is also observed that the report generation component automatically adapts the format of the assessment report it produces based on what organization completes the assessment. For example, if a first hospice care organization that uses a first assessment template completes a first assessment and a second hospice care organization that uses a second assessment template completes a second assessment, when the report generation component generates a first assessment report based on the first completed assessment and generates a second assessment report based on the second completed assessment, the reports manifest different formats.

Some assessment format changes are adopted by the report generation utility library by revising mapping of identities to text names, for example revising mapping of section identities to section text names and/or revising mapping of card identities to card text names. In an embodiment, the mapping of identities to text names may be provided by an identity-to-name mapping JSON in the library, and thus changing the identity to text name mapping may be accomplished by editing the JSON. In another embodiment, the mapping of identities to text names may be defined in a data store, and thus changing the identity to text name mapping may be accomplished by revising the data store, for example changing an identity-to-text name mapping table. Some assessment format changes are adopted by the report generation component by adding a new card-type specific method to the report generation utility library and a corresponding new card-type specific sub-report component to the report generation tool. It is noted that adding a new card type is expected to be a rare event. This design approach of the report generation component—customizing a third party report generation tool with a report generation utility library where common changes are restricted to editing data in a JSON (or revising the identity-to-text name table in the data store)—makes the report generation component very flexible and robust to change. This design approach also supports the system goal of efficiency in that assessment format changes are easy to implement and typically are restricted to editing data values in the report generation utility library.

Many reporting tools are implemented as a statically coded tool that is configured to operate with a static interface, for example a statically defined traditional data set in a data store. Said in other words, often reporting tools are configured to operate with a statically defined data base schema. If the data base schema changed, the reporting tool ordinarily would need to be reconfigured and/or recoded to interwork with the changed schema. This increased the level of effort involved in changing systems, making the systems resistant to change. This was a problem, too, when supporting different assessment format preferences of different hospice care or palliative care organizations. In the patient care giver tool system taught herein, this short-coming of traditional reporting tools might have been exacerbated, since this system promotes increased flexibility of changing assessment formats. By making assessment format changes easier, it is expected that the system will accelerate assessment format changes, putting the spotlight on the limitations of traditional reporting tools. By flowing the definition of assessment formats along with assessment input data, and by establishing the library of report generation utilities to make the report generation component more flexible, this problem was overcome.

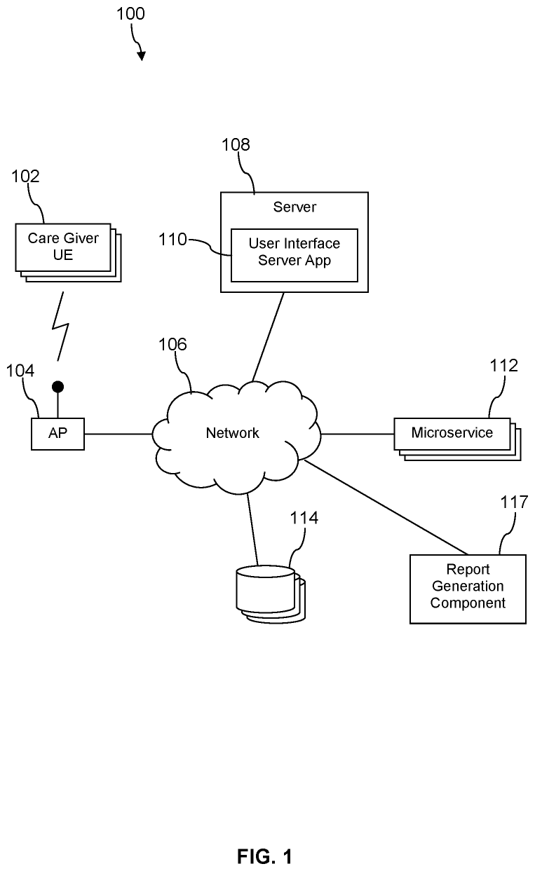

Turning now to FIG. 1, a patient care giver tool system 100 is described. In an embodiment, the system 100 comprises a plurality of user equipments (UEs) 102, a wireless access point (AP) 104, a network 106, a server 108 executing a user interface server application 110, a plurality of microservices 112, a plurality of data stores 114, and a patient assessment report generation component 117. The UEs 102 may be used by patient care givers to access a patient care tool user interface extended by the user interface server application 110. The patient care tool interface provides access to the microservices 112 and functionality that they support.

The UEs 102 may be tablet computers, notebook computers, laptop computers, mobile communication devices, or wearable computers. The network 106 comprises one or more public communication networks, one or more private communication networks, or a combination thereof. The wireless AP 104 may be a WiFi access point. The wireless AP 104 may be a cell site, such as a cell tower. In an embodiment, each of the microservices 112 may be provided by modules or applications that execute on server computers. In an embodiment, two or more of the microservices 112 may be hosted by the same server computer. In an embodiment, the user interface server application 110 may execute on the same server that hosts one or more of the microservices 112. In an embodiment, the data stores 114 may be provided by database management systems (DBMSs) executing on computer systems and providing mediated access to data storage. In an embodiment, the user interface server application 110, the microservices 112, and the data stores 114 may be provided by a cloud computing environment. In a cloud computing environment, the microservices 112 may execute in a multi-tenant mode of operation (e.g., serving a plurality of hospice care organizations at the same time).

The care giver UE 102 may present a user interface to the patient care giver tool system 100 that comprises a plurality of different views into the system 100. For example, the user interface on the care giver UE 102 may present an assessment section in a right hand side of the display, a plurality of assessment icons in a left hand side of the display, where the assessment icons promote selection of an assessment section, and a plan of care in a middle of the display. The plan of care may identify a patient name, a date of birth, an age, a primary diagnosis, and a secondary diagnosis. The plan of care may further provide a residential address of the patient, a level of care of the patient, a list of known allergies of the patient, a list of current medications of the patient, a case manager, an attending physician, and various care instructions. As the care giver completes sections of an assessment, he or she may save the assessment section. On selection of the save, the user interface server application 110 sends the data from the section to the assessment microservice 112, and the assessment microservice 112 stores both the input data and data defining the format of the section to a data store 114, for example in a JSON format in a JSON-B column of an entry in a Postgres database. In an embodiment, assessment sections may comprise one or more of a vitals section, a neurological section, a pain section, a respiratory section, a cardiovascular section, a gastrointestinal section, a genitourinary section, a nutrition/metabolic section, a skin section, a musculoskeletal section, an ADLS/functional needs section, a precautions and safety section, a summary section, a face-to-face section, a supervisory visit section, and a hospice aide section.

The assessment report generation component 117 may access one or more of the data stores 114 to access a completed patient assessment entry. The assessment report generation component 117, in an embodiment, may be provided as a microservice 112. The assessment report generation component 117 may comprise a third party report generation software tool, such as Jasper report generation software. In an embodiment, the assessment report generation component 117 comprises a third party report generation software tool that links in a report generation utility library that comprises configuration data and methods that have the effect of customizing the third party report generation software tool for use in the system 100. In an embodiment, the assessment generation component 117 is not natively configured to interwork with all the formats used in the assessment templates and formatted as a user interface document by the user interface server application 110. In an embodiment, the assessment report generation component 117 uses the report generation utility library to read the assessment template information from the completed patient assessment entry, to read the assessment input data, and to transcode both the template information and the assessment input into the form of Java objects amenable to processing by the third party report generation software tool. The report generation utility library transforms some information such as time information into a format more useful for a report to be read by human beings. For example, the report generation utility library transforms time date stamps recorded in a coordinated universal time (UTC) representation system to a local time representation system associated with where the assessment was completed.

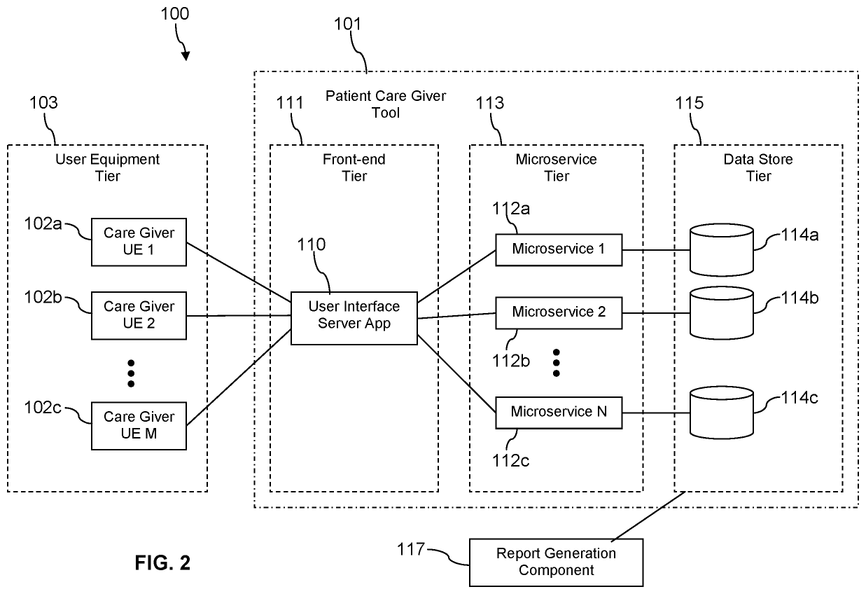

Turning now to FIG. 2, a different depiction of the patient care giver tool system 100 is described. While the depiction of system 100 in FIG. 1 illustrates communication linkages among elements of the system 100, the depiction of system 100 in FIG. 2 illustrates the three-tiered architecture and the functional relationships among the elements of the system 100. The patient care giver tool 101 comprises a front-end tier 111 comprising the user interface server application 110, a microservice tier 113 comprising a first microservice 112a, a second microservice 112b, and a third microservice 112c, and a data store tier 115 comprising a first data store 114a, a second data store 114b, and a third data store 114c.

The patient assessment report generation component 117 is illustrated as separate from the microservice tier 113 but columnarly aligned with the microservice tier 113. The patient assessment report generation component 117 can be considered to be a part of the three-tier architecture of the patient care giver tool 101 that is similar to that of the microservice tier 113, although in an embodiment the patient assessment report generation component 117 is not deployed as a microservice. In a different embodiment, however, the patient assessment report generation component 117 may be deployed as a microservice. Like the microservices 112, the patient assessment report generation component 117 is intermediate between the front end tier 111 and the data store tier 115.

The system 100 further comprises what may be referred to as a user equipment tier 103 that comprises a first care giver UE 102a, a second care giver UE 102b, and a third care giver UE 102c. While three UEs 102 are illustrated in FIG. 2, it is understood that the UE tier 103 may comprise any number of UEs 102. While three microservices 112 are illustrated in FIG. 2, it is understood that the microservice tier 113 may comprise a greater number of microservices 112. For example, in an embodiment, the microservice tier 113 comprises an assessment microservice, a plan of care microservice, an interdisciplinary group (IDG) microservice, an alerts and notifications microservice, a patient referral microservice, and a billing microservice. While three data stores 114 are illustrated in FIG. 2, it is understood that the data store tier 115 may comprise any number of data stores. In an embodiment, the data store tier 115 comprises an assessment data store, a plan of care data store, an IDF data store, and an alerts and notifications data store. In an embodiment, the microservices 112 execute in a cloud computing environment in a multi-tenant mode of operation serving a plurality of patient care organizations, hospice care organizations, or palliative care organizations.

In an embodiment, the data stores 114 may be provided by a single data base management system (DBMS) and the data stores 114a, 114b, 114c may be independent sets of tables, where each independent set of tables is governed by its own database schema. In an embodiment, the data stores 114 are provided as a PostgreSQL DBMS. PostgreSQL is often referred to alternatively as Postgres. In an embodiment, the user interface server application 110 is developed as or based on an Angular application and an Ionic application. Angular is a TypeScript-based front-end development platform. Ionic is an HTML5 mobile application interface framework. Ionic is used to provide user interface look-and-feel presentation associated to the specific operating system of the UEs 102 (e.g., an Android look-and-feel for an Android UE operating system and an iOS look-and-feel for an Apple UE operating system).

When a hospice organization (or, alternatively, a palliative care organization) desires to configure an assessment section, card, or field, they may modify a template defining the format of the assessment in the data store 114 associated with the assessment microservice 112. This may entail editing a JSON document and storing it in a table of the data store 114, for example, in a JSON-B column of a Postgres database. Likewise, if the hospice organization desires to change the role based association of assessment sections, this can be accomplished by editing a JSON document and storing it in a table of the data store 114.

When the assessment template or the role based associations of the assessment template has been modified, the next time an assessment user interface is requested by the UE 102 from the user interface server application 110, the changed assessment format is used, without recoding, without recompiling, without generating a new schema. Each time a UE 102 selects a different assessment section for display on the UE 102, the subject assessment section is requested from the user interface server application 110. The user interface server application 110 requests the subject assessment section via the RESTful API extended by the assessment microservice 112. The assessment microservice 112 requests the appropriate assessment section template from the data store 114 associated with the assessment microservice 112.

The assessment microservice 112 parses the JSON document containing the desired template information and constructs an assessment section document based on the JSON document and based on the role associated with the UE 102. Because the template information retrieved from the data store 114 contains the changed assessment format and/or the changed role based associations of the assessment template, the assessment section document that the assessment microservice 112 constructs reflects the modification of the assessment template. As an example of role based access control, a nurse may be able to see a vitals assessment section, a neurological assessment section, a pain assessment section, and other assessment sections on his UE 102; but a hospice aid may be able to see a vitals assessment section but not be able to see a neurological assessment section or a pain assessment section on his UE 102. Note that the template does define these assessment sections, but the care giver role associated to the care giver (via the UE 102 request to the user interface server application 110 that contains the identity of the user which is itself mapped to a care giver role) is used to restrict what assessment sections may be seen on the UEs 102.

The assessment microservice 112 returns the constructed assessment section document via the RESTful API to the user interface server application 110. The user interface server application 110 adapts the constructed assessment section document for presentation on the UE 102 and sends the adapted assessment section document to the UE 102. The UE 102 then presents the subject assessment section in its display.

When the user inputs data into an assessment section and saves the section on the UE 102, the input data is returned to the user interface server application 110. The user interface server application 110 sends the input data via the RESTful API to the assessment microservice 112. The assessment microservice 112 saves the assessment section input data as well as the template format data associated with the assessment section in a new row in an assessment table, for example as JSON human readable attribute-value pairs in a JSON-B column of the assessment table. The assessment table may comprise other columns that are statically defined in a database schema, for example a patient identity column, a hospice organization identity column, a date and time column. The assessment microservice 112 populates appropriate values into these statically configured columns in the new entry in the assessment table.

When a section entry has been stored in the assessment table, in an embodiment the assessment microservice 112 launches a thread that requests the data for the assessment section just stored in the data store 114 and launches a separate thread that requests all scripts defined in the data store 114 for the subject assessment section. The assessment microservice 112 executes each script in turn. The scripts each evaluates one of the items of data stored in the assessment section against a criterion. If the value of the data item satisfies the criterion, the script creates an issue that it attaches to the plan of care associated with the patient. For example, a script may define a criterion that if the patient weight is 250 LBS or more, an issue should be attached to the patient plan of care.

In an embodiment, the scripts are programmed using the Groovy scripting language. Some of the microservices 112 are at least partially developed in the Java programming language, and Groovy is a scripting language that integrates smoothly with Java programs. In another embodiment, however, a different scripting language may be used to program the scripts, for example Perl or Ruby. The assessment microservice may be developed within a Spring Boot programming framework which automatically builds the RESTful API code extended for use by the assessment microservice 112 to the user interface server application 110. The Spring Boot programming framework simplifies developing a new application, relieving developers of creating repetitive structures, for example RESTful API code support.

When the user of the UE 102 completes an assessment and saves, the assessment microservice 112 accesses all the entries in the assessment table associated with that assessment (e.g., a row entry for each section of the assessment), creates a new entry or row comprising the input data and the format or assessment template data from all the sections, and stores the new entry in a completed assessment table. This completed assessment table may be said to store completed assessments, for example completed hospice care patient assessments and/or palliative care patient assessments. In an embodiment, the assessment report generation component 117 processes the new entry to create a report representing the completed assessment, for example a PDF file or a byte stream capturing the results of the completed assessment. Because the assessment report generation component 117 may not be capable of understanding the assessment template format information portion of the completed assessment entries, in an embodiment, the assessment report generation component 117 uses a library of report generation utilities to read the assessment template portion of the completed assessment entries and to transcode the assessment input into Java objects amenable for processing by the report generation tool. In an embodiment, Jasper report generation software is used to generate the report based on the entry in the completed assessment table.

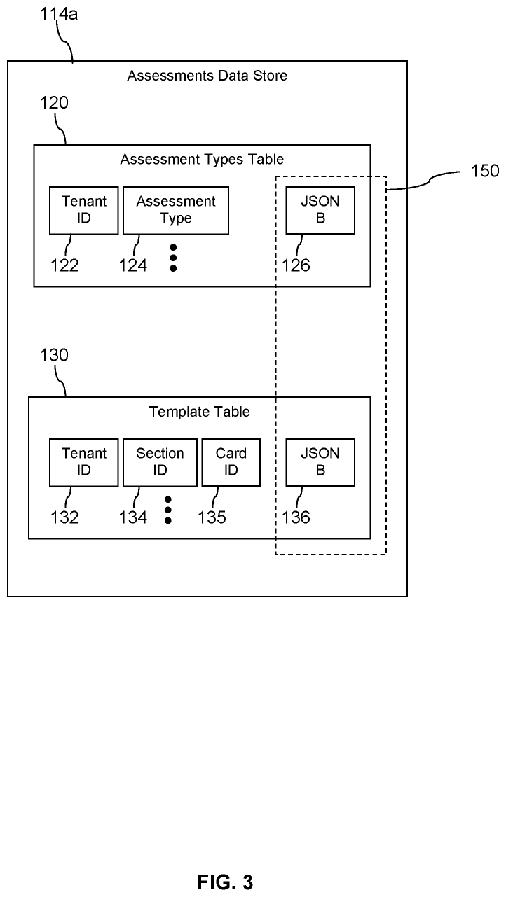

Turning now to FIG. 3, exemplary data store tables are described. In an embodiment, the data store tier 115 comprises an assessments data store 114a that defines assessment templates used to provide an assessment form to be presented on the UE 102. The assessments data store 114a comprises an assessment types table 120 and a template table 130. Each row in the assessment types table 120 defines a format of an assessment type 124, for example in an assessment format JSON B column 126.

Example assessment types comprise an initial comprehensive assessment type, a comprehensive assessment type, a routine visit assessment type, and other assessment types. In some circumstances the assessment types table 120 may comprise a plurality of rows associated with the same assessment type, for example a plurality of rows defining different formats of initial assessment types. The different rows associated with the same assessment type 124 are distinguished by having different tenant IDs 122. A default definition of a format of an assessment type 124 may be created as a row in the assessment types table 120, for example a row having a tenant ID of a null value, a zero value, or some other distinctive value that distinguishes it from a specific hospice organization. If a hospice organization wishes to define a custom assessment format for an assessment type 124 that deviates from the default format for the assessment type 124, a row may be added to the assessment types table 120 that identifies the assessment type 124, the tenant ID 122 (e.g., a unique identifier associated to the hospice organization), and defines the custom assessment format in the assessment format JSON B column 126. The assessment format JSON B column 126 may define the format of the assessment type as a plurality of sections that compose the assessment form.

Each row in the template table 130 defines a section format or a card format, for example in a format JSON B column 136. Each row in the template table 130 comprises a section identity 134 and a card identity 135. If the row defines a template or format for a section, the card identity 135 stores a null value. If the row defines a template or format for a card, the section identity 134 identifies a section that contains the card, and the card identity 135 identifies the card.

If the row in the template table 130 defines a section format, the format JSON B column 136 may define the format of the section as a plurality of cards that compose the section. In some circumstances, the template table 130 may comprise a plurality of rows associated with the same section identity 134 and that store a null value in the card identity 135 (e.g., the row defines a section format) but having different tenant IDs 132. A default definition of a format for a section identity 134 may be created as a row in the template table 130 having a tenant ID 132 that is distinctive value such as a null value or a zero value. If a hospice organization wishes to define a custom section format for a section identity 134 that deviates from the default format for the section identity 134, a row may be added to the template table 130 that identifies the section identity 134, the card identity 135 being a null value, the tenant ID 132, and defining the custom section format in the format JSON B column 136.

Each row in the template table 130 that contains a non-null section identity 134 and a non-null card identity 135 and defines a format of a card, for example in the format JSON B column 136. The format JSON B column 136 may define the format of the card as a plurality of fields that compose the card and further defines the layout of the fields. In some circumstances, the template table 130 may comprise a plurality of rows associated with the same card identity 135. The different rows associated with the same card identity 135 are distinguished by having different section identities 134 and/or different tenant IDs 132. A default definition of a format for a card identity 135 may be created as a row in the template table 130 having a tenant ID having a null value, a zero value, or another distinctive value. If a hospice organization wishes to define a custom card format that deviates from the default card format defined for the card identity 135, a row may be added to the template table 130 that identifies the section identity 134, the card identity 135, the tenant ID 132, and defining the custom card format in the format JSON B column 136.

The assessment type format JSON B column 126 and the format JSON B column 136 associated with a given hospice organization may be viewed as an assessment format template 150 associated to that given hospice organization. It is understood that the assessment format template 150 may comprise some default format template components as well as some custom format template components. In some contexts, the format JSON B 136 associated with a specific section ID 134 and a specific tenant ID 132 may be viewed as an assessment section format template or a section template. In some contexts, the format JSON B 136 associated with a specific card ID 135 and a specific tenant ID 132 may be viewed as an assessment card format template or a card template. In another embodiment, the assessment format template 150 may be stored in a different way. In an embodiment, the assessments data store 114a comprises additional tables and data that are not illustrated in FIG. 3, for example tables storing information on completed sections (e.g., input data and section template information, as described further above), tables storing completed assessments, and other data. In an embodiment, the format information for sections and card may be stored differently, for example with section format information stored in a section format table and with card format information stored in a card format table.

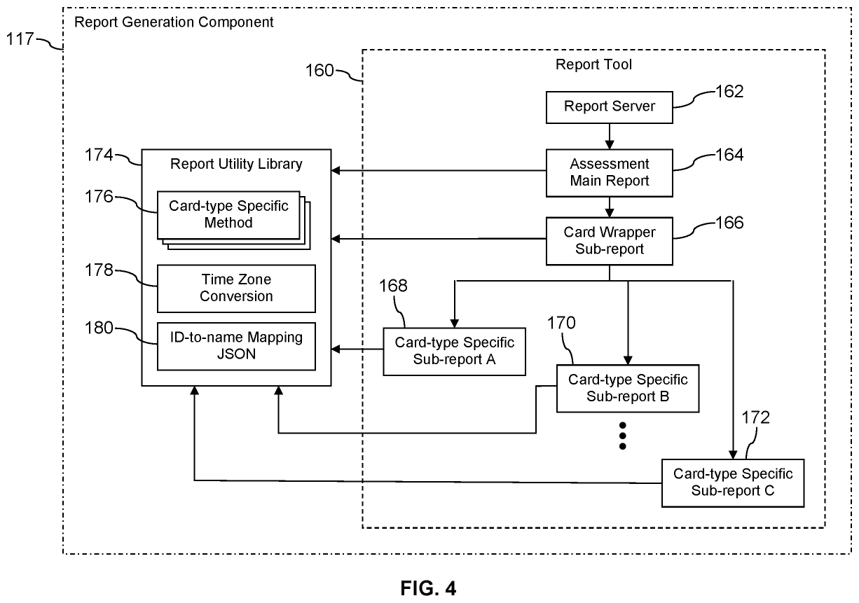

Turning now to FIG. 4, further details of the report generation component 117 are described. In an embodiment, the report generation component 117 comprises a report generation tool 160 and a report generation utility library 174. The report generation tool 160 may link or otherwise access data and methods encapsulated by the report generation utility library 174. In an embodiment, the report generation utility library 174 is provided as a Java archive (JAR) file. In an embodiment, the report generation tool 160 comprises a report server 162, an assessment main report 164, a card wrapper sub-report 166, and a plurality of card-type specific sub-reports, for example, a first card-type specific sub-report A 168, a second card-type specific sub-report B 170, and a third card-type specific sub-report C 172. Each of the components 162, 164, 166, 168, 170, 172 may be implemented as sub-components, sub-routines, or scripts. In some contexts, the card-type specific sub-reports 168, 170, 172 may be referred to as card-type specific sub-report components. In an embodiment, the report generation utility library 174 comprises a plurality of card-type specific methods 176, a time zone conversion method 178, and an identity-to-name mapping JSON 180. The identity-to-name mapping JSON 180 defines associations of section identities to section text names and defines associations of card identities to card text names. As an example, a section identity of “EL0013” may be mapped to a section text name of “Neurological.” Alternatively, in an embodiment, the data store 114 comprises an identity-to-name mapping table that defines the associations of section identities to section text names and defines the associations of card identities to card text names. In this alternative embodiment, the report generation component 117 reads the identity-to-name mapping table from the data store 114 when it is initiated or launched to learn the associations of identities to names.

In an embodiment, when an assessment report is completed, the assessment microservice 112 invokes the report generation component 117, for example by invoking the report server 162 while passing an assessment report identity. The report server 162 invokes the assessment main report 164 while passing the assessment report identity. The assessment main report 164 performs a Postgres query against the assessments data store 114a with the assessment report identity. The response to the Postgres query, assuming the data store look-up is successful, comprises JSON template information and JSON assessment input information. In an embodiment, the assessment main report 164 performs a series of Postgres queries against the assessment data store 114a, for example one query for each of a plurality of cards in the subject assessment. In addition to JSON template and JSON assessment input information, the returned information may comprise a section identity, a sort order of the section, a type of the card, a card identity, a sort order of the card within the section.

The assessment main report 164 invokes a method in the report generation utility library 174 to map the section identity to a textual representation of the section name (e.g., a string value). The report generation utility library 174 looks up the section text name based on the section identity using the ID-to-name mapping JSON 180. The assessment main report 164 invokes a method in the report generation utility library 174 to map the card identity to a textual representation of the card name (e.g., a string value). The report generation utility library 174 looks up the card text name based on the card identity using the ID-to-name mapping JSON 180. The assessment main report 164 passes the section text name, the card text name, and the other information retrieved from the assessments data store 114a to the card wrapper sub-report 166.

The card wrapper sub-report 166 identifies a card-type specific sub-report 168, 170, 172 based on the type of card and passes the section text name, the card text name, and the other information retrieved from the assessments data store 114a to that card-type specific sub-report 168, 170, 172. In some cases, depending on the type of card, the card wrapper sub-report 166 may process some of the information and add this to the information it passes to the card-type specific sub-report 168, 170, 172. For example, if the card type is a list card type, the card wrapper sub-report 166 passes the JSON assessment input information to the report generation utility library 174 and receives back a number of headers included in the list card. The card wrapper sub-report 166 then passes this additional information to the card-type specific sub-report 168, 170, 172.

The card-type specific sub-report 168, 170, 172 sends the information (e.g., the JSON template information, the JSON assessment input information, the section identity, the sort order of the section, the type of the card, the card identity, the sort order of the card within the section) to a card-type specific method 176 of the report generation utility library 174. The card-type specific method 176 converts the information to one or more Java objects that are amenable for processing by the report generation tool 160, for example by the report server 162 and/or by the assessment main report 164. The card-type specific method 176 may convert time values in the JSON assessment input from UTC to local time representations, for example by invoking the time zone conversion method 178. The card-type specific method 176 may convert some text values to a fuller text result. The card-type specific method 176 may format the content of the Java objects to be more aesthetically pleasing or readable by a human reader (e.g., putting text into pretty printing format).

In an embodiment, the card-type specific method 176 creates the Java objects at least in part based on the associations of section identities to section text names and based on the associations of card identities to card text names defined by the identity-to-name mapping JSON 180. Hence, by changing or adding to the associations defined by the identity-to-name mapping JSON 180, the card-type specific methods 176 may change the Java objects which they create, without the card-type specific methods 176 having themselves been recoded or altered. When an assessment template is changed to change and/or to add a new section name or a new card name, the identity-to-name mapping JSON 180 may be edited to reflect this change in the assessment template. Alternatively, in an embodiment, identity-to-name mapping may be defined by an identity-to-name mapping table in the data store 114, and revising the identity-to-name mapping may be accomplished by revising the identity-to-name mapping table.

The card-type specific method 176 then returns the one or more Java objects to the card-type specific sub-report 168, 170, 172. It is understood that what is returned to the card-type specific sub-report 168, 170, 172 is useable by the report generation tool 160 to produce a portion of an assessment report as a PDF or as a byte stream, for example a card of the assessment report. The series of processing of cards in this manner aggregates to produce a complete assessment report.

When changes to assessment formats occur, changes to the report generation component 117 are typically limited in scope or possibly unnecessary. If a new card is added to a section that is of a predefined card type, the name and card identity of the new card is added to the ID-to-name mapping JSON 180. If a new section is added, the name and section identity of the new section is added to the ID-to-name mapping JSON 180. It may be that new cards are also defined in the new section, and in that case the name and card identities of those new cards would need to be added to the ID-to-name mapping JSON 180 also. It is noted that the changes associated with adding a new section and a new card of a predefined card type are data configuration changes only (e.g., changes in the ID-to-name mapping JSON 180) and hence of limited repercussions and complexity. If a new card type is added, a new card-type specific sub-report may be added, and a reference to the new card-type specific sub-report may be added to the card wrapper sub-report 166.

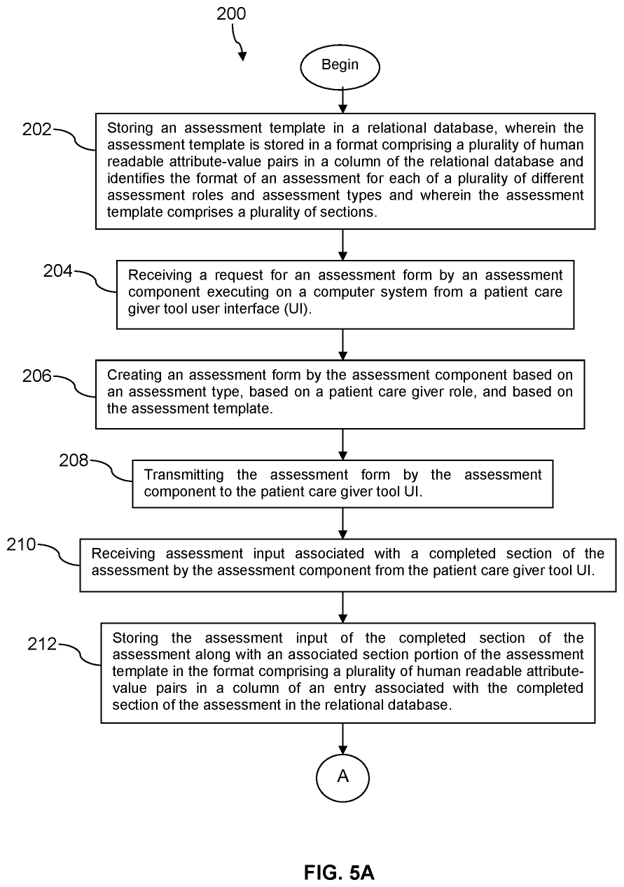

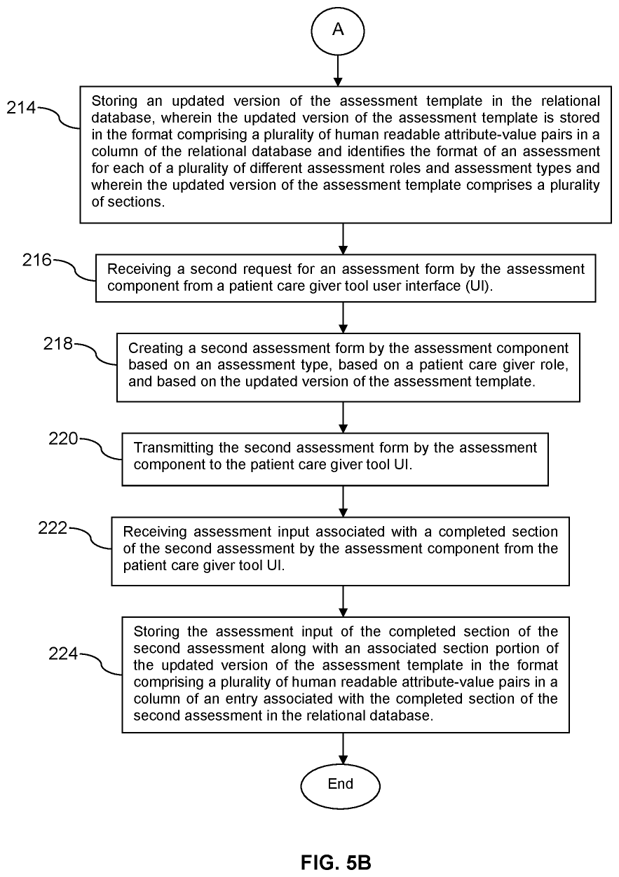

Turning now to FIG. 5A and FIG. 5B, a method 200 is described. In an embodiment, the method 200 is a method of dynamically constructing a patient assessment user interface using a three-tier architecture. The three-tier architecture is best seen in FIG. 2, where the three tiers are (A) a front-end tier 111, (B) a microservice tier 113, and (C) a data store tier 115. It is noted that this three-tier architecture supports the efficiency and flexibility goals established for the patient care giver tool 101.

At block 202, the method 200 comprises storing an assessment template in a relational database, wherein the assessment template is stored in a format comprising a plurality of human readable attribute-value pairs in a column of the relational database and identifies the format of an assessment for each of a plurality of different assessment roles and assessment types and wherein the assessment template comprises a plurality of sections. In an embodiment, the format comprising a plurality of human readable attribute-value pairs is a JSON format. In an embodiment, the relational database is the database 114 in the data store tier 115. In an embodiment, the relational database is a PostgreSQL database (in some contexts PostgreSQL is referred to as a Postgres database). The assessment template stored during block 202 may be an initial assessment template established on first deploying the patient care giver tool 101. Alternatively, the assessment template stored during block 202 may be a revised assessment template, created by text editing the human readable attribute-value pairs, adding one or more human readable attribute-value pairs, and/or deleting one or more human readable attribute-value pairs.

The human readable attribute-value pairs may define sections, cards, and/or fields of the assessment form, for example defining section layouts in terms of cards, card layouts in terms of fields, field layout in terms of data types and data size or length. The human readable attribute-value pairs may define text to be displayed in sections, cards, and/or fields, for example names of sections, cards, and/or fields.

At block 204, the method 200 comprises receiving a request for an assessment form by an assessment component executing on a computer system from a patient care giver tool user interface (UI). In an embodiment, the patient care giver tool UI is the user interface server application 110. The assessment component may receive the request for the assessment form via a REST application programming interface (API) that it extends to the patient care giver tool UI. The request may identify a patient care giver role of the user of the UE 102 initiating an assessment. The request may identify an assessment type. The patient care giver role of the user of the UE 102 may be determined based on a login of the user. For example, every user granted a login to the patient care giver tool 101 may be predefined to be associated to a specific patient care giver role, e.g., one of a nurse role, a doctor role, a spiritual advisor role, or a social worker role.

At block 206, the method 200 comprises creating an assessment form by the assessment component based on an assessment type, based on a patient care giver role, and based on the assessment template. The assessment component may execute role based access control rules to select sections from a generic assessment template for creating the assessment form. Likewise, the assessment component may select some but not all of the sections from the generic assessment template based on the assessment type. In an embodiment, the processing of block 206 may further be based on an identity of a hospice organization and/or on a tenant ID. In an embodiment, the processing of block 206 may further be based on a level of care associated with the patient. In an embodiment, levels of care comprise routine home care, continuous care, respite care, and general in-patient care. In another embodiment, either fewer or more levels of care may apply.

At block 208, the method 200 comprises transmitting the assessment form by the assessment component to the patient care giver tool UI. In an embodiment, the assessment component transmits the assessment form via the REST API to the patient care giver UI. At block 210, the method 200 comprises receiving assessment input associated with a completed section of the assessment by the assessment component from the patient care giver tool UI. In an embodiment, the patient care giver tool UI sends the assessment input via the REST API. The assessment input may have been input by a user of the UE 102 and a save button in an assessment section screen may have been selected or the user may have navigated from a first assessment section display to a second assessment display on the UE 102.

At block 212, the method 200 comprises storing the assessment input of the completed section of the assessment along with an associated section portion of the assessment template in the format comprising a plurality of human readable attribute-value pairs in a column of an entry associated with the completed section of the assessment in the relational database. In an embodiment, the assessment input and the associated section portion of the assessment template are stored in JSON format in a JSON-B column of the relational database. In an embodiment, the assessment input and the associated section portion of the assessment template are stored in a new row entry in the relational database, where some information in the new row entry is formatted in statically structured columns of the new row entry and the assessment information and associated section portion of the assessment template are stored in an unstructured column of the new row entry. The statically structured columns of the new row entry may identify a patient, a hospice care organization, and a time and date.

At block 214, the method 200 comprises storing an updated version of the assessment template in the relational database, wherein the updated version of the assessment template is stored in the format comprising a plurality of human readable attribute-value pairs in a column of the relational database and identifies the format of an assessment for each of a plurality of different assessment roles and assessment types and wherein the updated version of the assessment template comprises a plurality of sections. The updated version of the assessment template stored during block 214 may be a revised assessment template, created by text editing the human readable attribute-value pairs, adding one or more human readable attribute-value pairs, and/or deleting one or more human readable attribute-value pairs. The assessment template may be updated by a hospice care organization or may be updated by a third party based on requirements defined by the hospice care organization. The assessment template may be updated to comply with additional regulatory requirements. The assessment template may be updated to improve a hospice care process of a hospice care organization.

At block 216, the method 200 comprises receiving a second request for an assessment form by the assessment component from a patient care giver tool user interface (UI). At block 218, the method 200 comprises creating a second assessment form by the assessment component based on an assessment type, based on a patient care giver role, and based on the updated version of the assessment template. In an embodiment, the processing of block 218 may further be based on an identity of a hospice organization and/or on a tenant ID. In an embodiment, the processing of block 218 may further be based on a level of care associated with the patient.

At block 220, the method 200 comprises transmitting the second assessment form by the assessment component to the patient care giver tool UI. It is understood that the second assessment form created based on the updated version of the assessment template is different in some way from the assessment form created in block 206. For example, the second assessment form may comprise one or more fields in an assessment section which were not present in the assessment form created at block 206. For example, the second assessment form may comprise a field that has a different input length than the same field in the assessment form created at block 206.

At block 222, the method 200 comprises receiving assessment input associated with a completed section of the second assessment by the assessment component from the patient care giver tool UI. At block 224, the method 200 comprises storing the assessment input of the completed section of the second assessment along with an associated section portion of the updated version of the assessment template in the format comprising a plurality of human readable attribute-value pairs in a column of an entry associated with the completed section of the second assessment in the relational database.

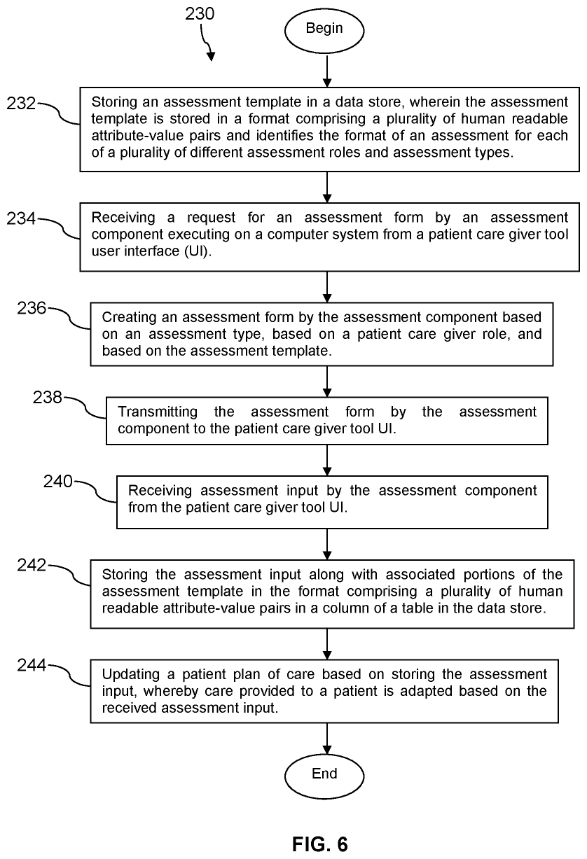

Turning now to FIG. 6, a method 230 is described. In an embodiment, the method 230 is an automated method of constructing a patient assessment user interface using a three-tiered architecture. At block 232, the method 230 comprises storing an assessment template in a data store, wherein the assessment template is stored in a format comprising a plurality of human readable attribute-value pairs and identifies the format of an assessment for each of a plurality of different assessment roles and assessment types.

At block 234, the method 230 comprises receiving a request for an assessment form by an assessment component executing on a computer system from a patient care giver tool user interface (UI). At block 236, the method 230 comprises creating an assessment form by the assessment component based on an assessment type, based on a patient care giver role, and based on the assessment template. In an embodiment, the processing of block 236 may further be based on an identity of a hospice organization and/or on a tenant ID. In an embodiment, the processing of block 236 is further based on a level of care associated with the patient. At block 238, the method 230 comprises transmitting the assessment form by the assessment component to the patient care giver tool UI.

At block 240, the method 230 comprises receiving assessment input by the assessment component from the patient care giver tool UI. At block 242, the method 230 comprises storing the assessment input along with associated portions of the assessment template in the format comprising a plurality of human readable attribute-value pairs in a column of a table in the data store. At block 244, the method 230 comprises updating a patient plan of care based on storing the assessment input, whereby care provided to a patient is adapted based on the received assessment input. For example, at block 244, an issue is attached to the plan of care. The plan of care is reviewed in an interdisciplinary group (IDG) and a goal is added to the plan of care based on the issue. Based on the newly added goal, an intervention is added to the plan of care. When a hospice care giver next conducts a visit and/or assessment of the subject patient, the care giver provides care based on the newly added intervention. For example, the care giver encourages the patient to listen to classical music to relieve their boredom and depression. For example, the care giver encourages the patient to drink more water. For example, the care giver boosts a pain medication dosage. For example, the care giver spends time to ask the patient to tell a story from his or her youth.

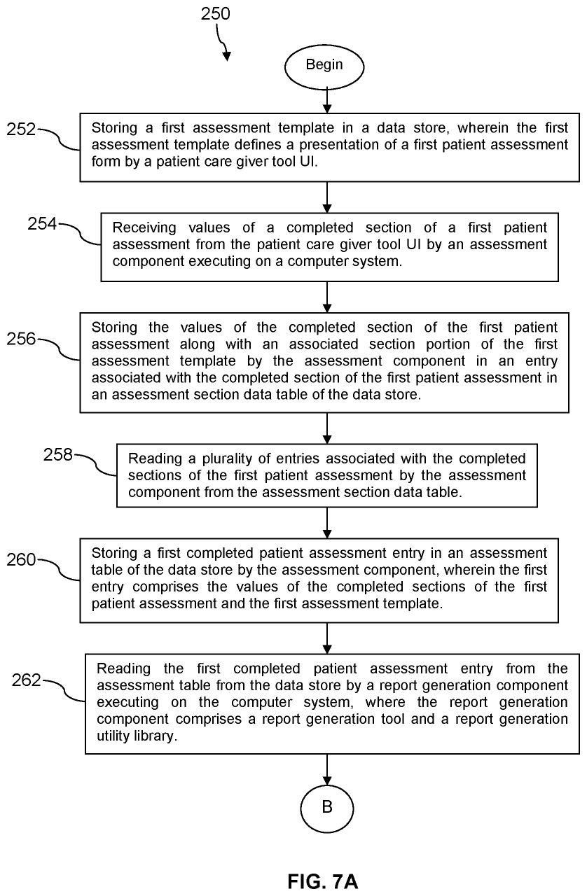

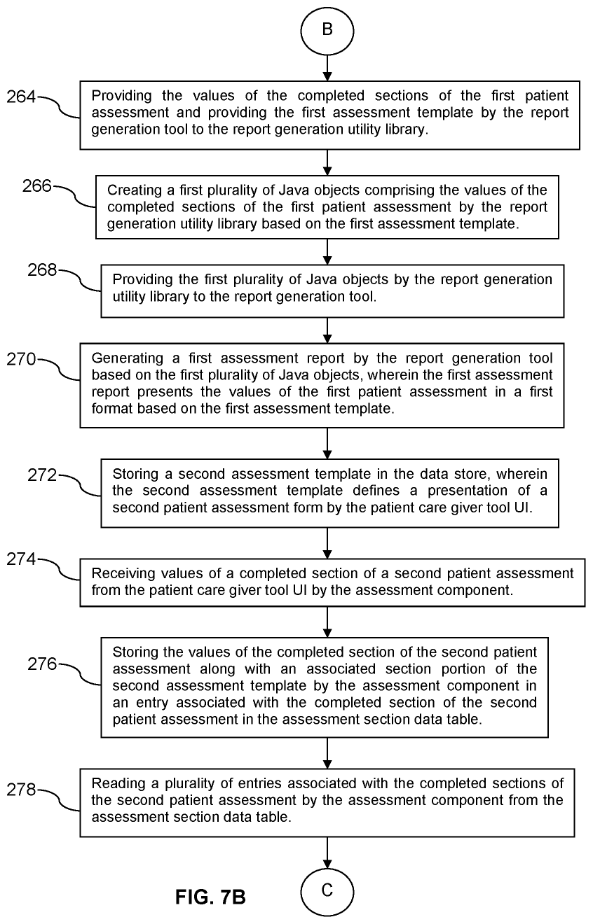

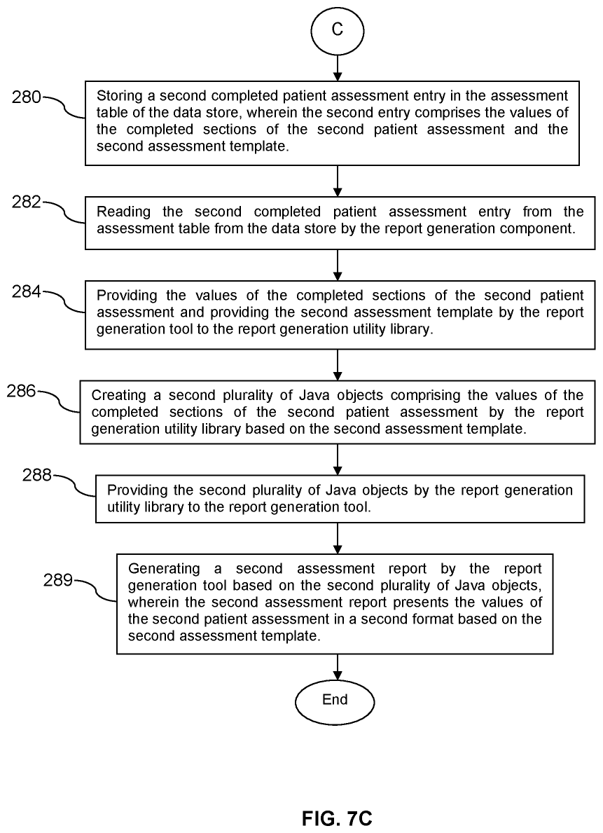

Turning now to FIG. 7A, FIG. 7B, and FIG. 7C, a method 250 is described. Method 250 is a method of using a three-tier architecture to dynamically generate a patient assessment report automatically adapted to an assessment format used in conducting a patient assessment. The patient assessment report may be a report on an assessment of a hospice care patient. The patient assessment report may be a report on an assessment of a palliative care patient. At block 252, the method 250 comprises storing a first assessment template in a data store, wherein the first assessment template defines a presentation of a first patient assessment form by a patient care giver tool UI. The assessment template may define a format or layout of a patient assessment as a set of sections, cards within the sections, and fields within the cards. The assessment template may define an arrangement of sections relative to each other, an arrangement of cards relative to each other within the sections, and an arrangement of fields relative to each other within the cards.

At block 254, the method 250 comprises receiving values of a completed section of a first patient assessment from the patient care giver tool UI by an assessment component executing on a computer system. At block 256, the method 250 comprises storing the values of the completed section of the first patient assessment along with an associated section portion of the first assessment template by the assessment component in an entry associated with the completed section of the first patient assessment in an assessment section data table of the data store.

At block 258, the method 250 comprises reading a plurality of entries associated with completed sections of the first patient assessment by the assessment component from the assessment section data table. At block 260, the method 250 comprises storing a first completed patient assessment entry in an assessment table of the data store by the assessment component, wherein the first entry comprises the values of completed sections of the first patient assessment and the first assessment template. The assessment template data that is stored in the completed patient assessment entry in assessment table may be accumulated from the template data associated with each of the saved sections and aggregated into a complete assessment template. At block 262, the method 250 comprises reading the first completed patient assessment entry from the assessment table from the data store by a report generation component executing on the computer system, where the report generation component comprises a report generation tool and a report generation utility library. The processing of block 262 may be performed mostly by the assessment main report component 164 of the report generation tool 160. It is noted that the processing of block 262 comprises reading the assessment template data from the assessment table.

At block 264, the method 250 comprises providing the values of the completed sections of the first patient assessment and providing the first assessment template by the report generation tool to the report generation utility library. At block 266, the method 250 comprises creating a first plurality of Java objects comprising the values of the completed sections of the first patient assessment by the report generation utility library based on the first assessment template. The processing of block 264 and block 266 may involve the assessment main report 164 invoking a method of the report generation utility library 174, the card wrapper sub-report component 166 invoking a method of the report generation utility library 174, and one or more card-type specific sub-report components 168, 170, 172 invoking corresponding card-type specific methods 176 of the report generation utility library 174.

At block 268, the method 250 comprises providing the first plurality of Java objects by the report generation utility library to the report generation tool. For example, one or more of the card-type specific methods 176 of the report generation utility library 174 provide one or more Java objects to the corresponding card-type specific sub-reports 168, 170, 172 of the report generation tool 160. The Java objects may be provided in the format of Java beans. In an embodiment, the Java objects may be provided in the format of one or more Java lists (e.g., lists of name, value pairs).

At block 270, the method 250 comprises generating a first assessment report by the report generation tool based on the first plurality of Java objects, wherein the first assessment report presents the values of the first patient assessment in a first format based on the first assessment template. The format of the first assessment report may comprise ordering sections relative to each other spatially, ordering cards relative to each other within sections spatially, and ordering fields relative to each other within cards spatially in accordance with the first assessment template. The format of the first assessment report may further comprise placing section titles, card titles, and field titles proximate to corresponding sections, cards, and fields.

At block 272, the method 250 comprises storing a second assessment template in the data store, wherein the second assessment template defines a presentation of a second patient assessment form by the patient care giver tool UI. The second assessment template may differ from the first assessment template in a variety of different ways. Sections may be rearranged relative to each other. Cards within sections may be rearranged within sections relative to each other. Fields within cards may be rearranged relative to each other. Formats of fields may be changed, for example shortened or lengthened. Sections may be added or removed. Cards may be added or removed. Fields may be added or removed. In an embodiment, the first assessment template may be associated with a first care organization having a first tenant identity, and the second assessment template may be associated with a second different care organization having a second different tenant identity. At block 274, the method 250 comprises receiving values of a completed section of a second patient assessment from the patient care giver tool UI by the assessment component.

In an embodiment, some of the changes to the assessment template (e.g., the changes reflected in the second assessment template) are reflected in the report generation utility library 174. For example, if section names are changed, if card names are changed, if new section names are added, and/or if new card names are added in the assessment template, the identity-to-name mapping JSON 180 may be edited to reflect those changes in the assessment template. If a new card type is added to the assessment template, a new card-type specific method 176 may be developed and added to the report generation utility library 174, and a new card-type specific sub-report component may be developed and added to the report generation tool 160. It is by making data changes to the identity-to-name mapping JSON 180 that many assessment template changes are accommodated by the report generation component 117. Alternatively, in an embodiment in which identity-to-name mapping is defined by an identity-to-name mapping table in the data store 114, it is by making data changes in the identity-to-name mapping table in the data store that many assessment template changes are accommodated by the report generation component 117. In some cases, however, assessment template changes do not entail changes either to the report generation utility library 174 or to the report generation tool 160 (e.g., if no new card type is added, if no new section is added, if no new card is added, if no section name is changed, and if no card name is changed). The structure of the report generation component 117 is designed to limit the scope of changes that result from the anticipated changes of assessment templates. Many changes entail only modifications of the data of the report generation utility library 174, for example the identity-to-name mapping JSON 180.

At block 276, the method 250 comprises storing the values of the completed section of the second patient assessment along with an associated section portion of the second assessment template by the assessment component in an entry associated with the completed section of the second patient assessment in the assessment section data table. At block 278, the method 250 comprises reading a plurality of entries associated with completed sections of the second patient assessment by the assessment component from the assessment section data table. At block 280, the method 250 comprises storing a second completed patient assessment entry in the assessment table of the data store, wherein the second entry comprises the values of completed sections of the second patient assessment and the second assessment template.

At block 282, the method 250 comprises reading the second completed patient assessment entry from the assessment table from the data store by the report generation component. At block 284, the method 250 comprises providing the values of the completed sections of the second patient assessment and providing the second assessment template by the report generation tool to the report generation utility library. At block 286, the method 250 comprises creating a second plurality of Java objects comprising the values of the completed sections of the second patient assessment by the report generation utility library based on the second assessment template. At block 288, the method 250 comprises providing the second plurality of Java objects by the report generation utility library to the report generation tool. At block 289, the method 250 comprises generating a second assessment report by the report generation tool based on the second plurality of Java objects, wherein the second assessment report presents the values of the second patient assessment in a second format based on the second assessment template.

In an embodiment, the processing of blocks 282, 284, 286, 288, and 289 are substantially similar to the processing of blocks 262, 264, 266, 268, and 270 described further above. The second assessment report is different, however, because the assessment template has changed with no change in the report generation component 117 or with change limited to data change in the identity-to-name mapping JSON 180 of the report generation utility library 174 (or, alternatively, data change in the identity-to-name mapping table in the data store 114) or with change limited to adding a card-type specific method 176 to the report generation utility library 174 and a card-type specific sub-report component to the report generation tool 160. It is noted that the first assessment template may be associated with a first organization and the second assessment template may be associated with a second organization. Thus, the differences between the first assessment template and the second assessment template may reflect different assessment presentation format preferences of different organizations. Alternatively, the first assessment template and the second assessment template may be associated with the same organization, where the second assessment template is an evolution of the earlier first assessment template. Assessment templates may evolve as organizations mature and develop and/or in response to government regulations.

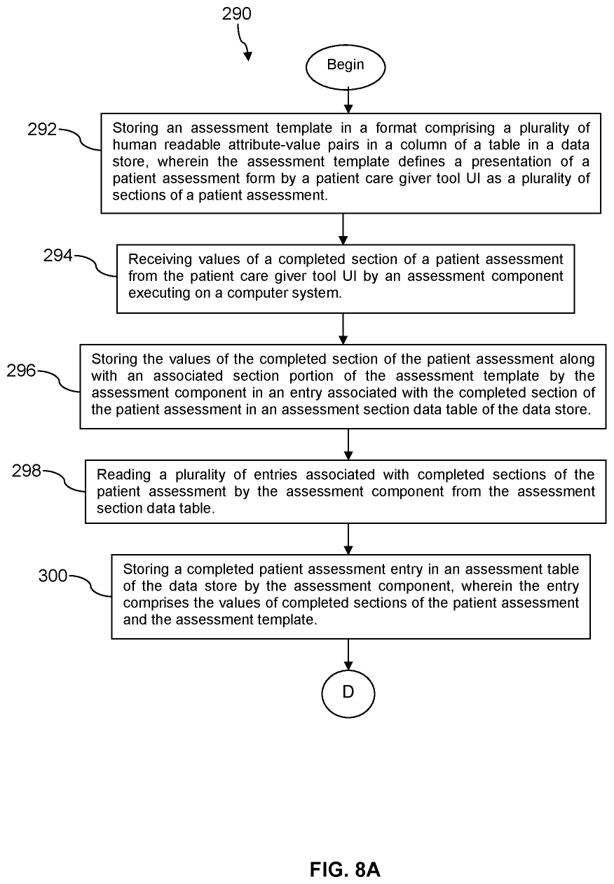

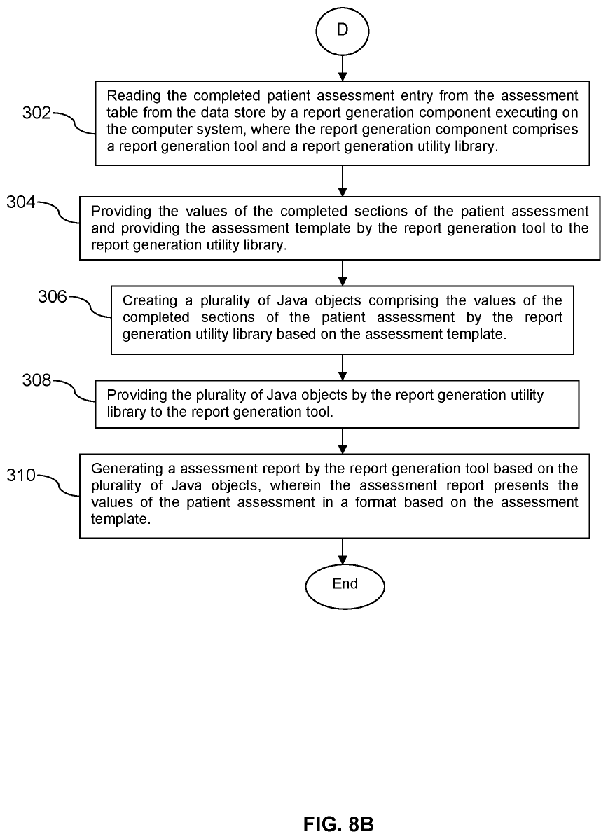

Turning now to FIG. 8A and FIG. 8B, a method 290 is described. The method 290 is a method of dynamically generating a patient assessment report automatically adapted to an assessment format used in conducting a patient assessment. At block 292, method 290 comprises storing an assessment template in a format comprising a plurality of human readable attribute-value pairs in a column of a table in a data store, wherein the assessment template defines a presentation of a patient assessment form by a patient care giver tool UI as a plurality of sections of a patient assessment. For example, the assessment template may be stored in JSON format. At block 294, method 290 comprises receiving values of a completed section of a patient assessment from the patient care giver tool UI by an assessment component executing on a computer system.

At block 296, method 290 comprises storing the values of the completed section of the patient assessment along with an associated section portion of the assessment template by the assessment component in an entry associated with the completed section of the patient assessment in an assessment section data table of the data store. At block 298, method 290 comprises reading a plurality of entries associated with completed sections of the patient assessment by the assessment component from the assessment section data table.

At block 300, method 290 comprises storing a completed patient assessment entry in an assessment table of the data store by the assessment component, wherein the entry comprises the values of completed sections of the patient assessment and the assessment template. At block 302, method 290 comprises reading the completed patient assessment entry from the assessment table from the data store by a report generation component executing on the computer system, where the report generation component comprises a report generation utility library.

At block 304, the method 290 comprises providing the values of the completed sections of the patient assessment and providing the assessment template by the report generation tool to the report generation utility library. At block 306, the method 290 comprises creating a plurality of Java objects comprising the values of the completed sections of the patient assessment by the report generation utility library based on the assessment template. At block 308, the method 290 comprises providing the plurality of Java objects by the report generation utility library to the report generation tool. At block 310, the method 290 comprises generating a assessment report by the report generation tool based on the plurality of Java objects, wherein the assessment report presents the values of the patient assessment in a format based on the assessment template.

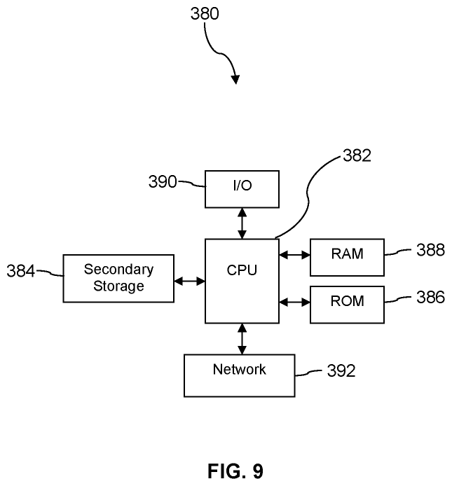

FIG. 9 illustrates a computer system 380 suitable for implementing one or more embodiments disclosed herein. The computer system 380 includes a processor 382 (which may be referred to as a central processor unit or CPU) that is in communication with memory devices including secondary storage 384, read only memory (ROM) 386, random access memory (RAM) 388, input/output (I/O) devices 390, and network connectivity devices 392. The processor 382 may be implemented as one or more CPU chips.

It is understood that by programming and/or loading executable instructions onto the computer system 380, at least one of the CPU 382, the RAM 388, and the ROM 386 are changed, transforming the computer system 380 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 380 is turned on or booted, the CPU 382 may execute a computer program or application. For example, the CPU 382 may execute software or firmware stored in the ROM 386 or stored in the RAM 388. In some cases, on boot and/or when the application is initiated, the CPU 382 may copy the application or portions of the application from the secondary storage 384 to the RAM 388 or to memory space within the CPU 382 itself, and the CPU 382 may then execute instructions that the application is comprised of. In some cases, the CPU 382 may copy the application or portions of the application from memory accessed via the network connectivity devices 392 or via the I/O devices 390 to the RAM 388 or to memory space within the CPU 382, and the CPU 382 may then execute instructions that the application is comprised of. During execution, an application may load instructions into the CPU 382, for example load some of the instructions of the application into a cache of the CPU 382. In some contexts, an application that is executed may be said to configure the CPU 382 to do something, e.g., to configure the CPU 382 to perform the function or functions promoted by the subject application. When the CPU 382 is configured in this way by the application, the CPU 382 becomes a specific purpose computer or a specific purpose machine.

The secondary storage 384 is typically comprised of one or more disk drives or tape drives and is used for non-volatile storage of data and as an over-flow data storage device if RAM 388 is not large enough to hold all working data. Secondary storage 384 may be used to store programs which are loaded into RAM 388 when such programs are selected for execution. The ROM 386 is used to store instructions and perhaps data which are read during program execution. ROM 386 is a non-volatile memory device which typically has a small memory capacity relative to the larger memory capacity of secondary storage 384. The RAM 388 is used to store volatile data and perhaps to store instructions. Access to both ROM 386 and RAM 388 is typically faster than to secondary storage 384. The secondary storage 384, the RAM 388, and/or the ROM 386 may be referred to in some contexts as computer readable storage media and/or non-transitory computer readable media.

I/O devices 390 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 392 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 392 may enable the processor 382 to communicate with the Internet or one or more intranets. With such a network connection, it is contemplated that the processor 382 might receive information from the network, or might output information to the network in the course of performing the above-described method steps. Such information, which is often represented as a sequence of instructions to be executed using processor 382, may be received from and outputted to the network, for example, in the form of a computer data signal embodied in a carrier wave.

Such information, which may include data or instructions to be executed using processor 382 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well-known to one skilled in the art. The baseband signal and/or signal embedded in the carrier wave may be referred to in some contexts as a transitory signal.

The processor 382 executes instructions, codes, computer programs, scripts which it accesses from hard disk, floppy disk, optical disk (these various disk based systems may all be considered secondary storage 384), flash drive, ROM 386, RAM 388, or the network connectivity devices 392. While only one processor 382 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 384, for example, hard drives, floppy disks, optical disks, and/or other device, the ROM 386, and/or the RAM 388 may be referred to in some contexts as non-transitory instructions and/or non-transitory information.

In an embodiment, the computer system 380 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 380 to provide the functionality of a number of servers that is not directly bound to the number of computers in the computer system 380. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing resources hired and/or leased from a third party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 380, at least portions of the contents of the computer program product to the secondary storage 384, to the ROM 386, to the RAM 388, and/or to other non-volatile memory and volatile memory of the computer system 380. The processor 382 may process the executable instructions and/or data structures in part by directly accessing the computer program product, for example by reading from a CD-ROM disk inserted into a disk drive peripheral of the computer system 380. Alternatively, the processor 382 may process the executable instructions and/or data structures by remotely accessing the computer program product, for example by downloading the executable instructions and/or data structures from a remote server through the network connectivity devices 392. The computer program product may comprise instructions that promote the loading and/or copying of data, data structures, files, and/or executable instructions to the secondary storage 384, to the ROM 386, to the RAM 388, and/or to other non-volatile memory and volatile memory of the computer system 380.

In some contexts, the secondary storage 384, the ROM 386, and the RAM 388 may be referred to as a non-transitory computer readable medium or a computer readable storage media. A dynamic RAM embodiment of the RAM 388, likewise, may be referred to as a non-transitory computer readable medium in that while the dynamic RAM receives electrical power and is operated in accordance with its design, for example during a period of time during which the computer system 380 is turned on and operational, the dynamic RAM stores information that is written to it. Similarly, the processor 382 may comprise an internal RAM, an internal ROM, a cache memory, and/or other internal non-transitory storage blocks, sections, or components that may be referred to in some contexts as non-transitory computer readable media or computer readable storage media.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method of using a three-tier architecture to dynamically generate a patient assessment report automatically adapted to an assessment format used in conducting a patient assessment, comprising:
storing a first assessment template in a data store, wherein the first assessment template defines a presentation of a first patient assessment form by a patient care giver tool UI;
receiving values of a completed section of a first patient assessment from the patient care giver tool UI by an assessment component executing on a computer system;
storing the values of the completed section of the first patient assessment along with an associated section portion of the first assessment template by the assessment component in an entry associated with the completed section of the first patient assessment in an assessment section data table of the data store;
reading a plurality of entries associated with the completed sections of the first patient assessment by the assessment component from the assessment section data table;
storing a first completed patient assessment entry in an assessment table of the data store by the assessment component, wherein the first entry comprises the values of the completed sections of the first patient assessment and the first assessment template;
reading the first completed patient assessment entry from the assessment table from the data store by a report generation component executing on the computer system, where the report generation component comprises a report generation tool and a report generation utility library;
providing the values of the completed sections of the first patient assessment and providing the first assessment template by the report generation tool to the report generation utility library;
creating a first plurality of Java objects comprising the values of the completed sections of the first patient assessment by the report generation utility library based on the first assessment template;
providing the first plurality of Java objects by the report generation utility library to the report generation tool;
generating a first assessment report by the report generation tool based on the first plurality of Java objects, wherein the first assessment report presents the values of the first patient assessment in a first format based on the first assessment template;
storing a second assessment template in the data store, wherein the second assessment template defines a presentation of a second patient assessment form by the patient care giver tool UI;
receiving values of a completed section of a second patient assessment from the patient care giver tool UI by the assessment component;
storing the values of the completed section of the second patient assessment along with an associated section portion of the second assessment template by the assessment component in an entry associated with the completed section of the second patient assessment in the assessment section data table;
reading a plurality of entries associated with the completed sections of the second patient assessment by the assessment component from the assessment section data table;
storing a second completed patient assessment entry in the assessment table of the data store, wherein the second entry comprises the values of the completed sections of the second patient assessment and the second assessment template;
reading the second completed patient assessment entry from the assessment table from the data store by the report generation component;
providing the values of the completed sections of the second patient assessment and providing the second assessment template by the report generation tool to the report generation utility library;
creating a second plurality of Java objects comprising the values of the completed sections of the second patient assessment by the report generation utility library based on the second assessment template;
providing the second plurality of Java objects by the report generation utility library to the report generation tool; and
generating a second assessment report by the report generation tool based on the second plurality of Java objects, wherein the second assessment report presents the values of the second patient assessment in a second format based on the second assessment template.

2. The method of claim 1, wherein the assessment table comprises hospice care patient assessments.

3. The method of claim 1, wherein the assessment table comprises palliative care patient assessments.

4. The method of claim 1, wherein the first and second assessment report each comprise a plurality of images of pain locations with reference to body parts.

5. The method of claim 1, wherein the report generation utility library comprises an identity-to-name mapping JSON that defines associations of section identities to section text names and associations of card identities to card text names, wherein creating the first plurality of Java objects and the second plurality of Java objects is based at least in part on the associations of section identities to section text names and on the associations of card identities to card text names.

6. The method of claim 5, further comprising editing the identity-to-name mapping JSON to add an association of a new section identity to a new section text name and to add an association of a new card identity to a new card text name, whereby the identity-to-name mapping JSON is amended to reflect a change in the second assessment template versus the first assessment template.

7. The method of claim 5, wherein the report generation utility library comprises a plurality of card-type specific methods, wherein creating the first plurality of Java objects and the second plurality of Java objects comprises the report generation tool invoking a plurality of the card-type specific methods and the invoked card-type specific methods creating the Java objects.

8. The method of claim 7, wherein the report generation tool comprises a plurality of card-type specific sub-report components, wherein each of the invoked card-type specific methods is invoked by a corresponding one of the card-type specific sub-report components and provides Java objects to its corresponding card-type specific sub-report component, and wherein each of the corresponding card-type specific sub-report components generates a corresponding card-specific part of the assessment reports.

9. The method of claim 8, further comprising:
adding a new card-type specific sub-report component to the report generation tool; and
adding a new card-type specific method to the report generation utility library,
whereby the report generation component is amended to reflect a change in the second assessment template versus the first assessment template.

10. A system for dynamically generating a patient assessment report adapted to an assessment format used in conducting a patient assessment, comprising:
at least one processor;
a data store;
a non-transitory memory;
an assessment component stored in the memory that, when executed by the at least one processor
stores a first completed patient assessment entry in an assessment table of the data store, wherein the first entry comprises values of a first patient assessment and a first assessment template that identifies the format of a presentation of a first patient assessment form by a patient care giver tool UI,
stores a second completed patient assessment entry in the assessment table of the data store, wherein the second entry comprises values of a second patient assessment and a second assessment template that identifies the format of a presentation of a second patient assessment form by the patient care giver tool UI and wherein the second assessment template is different from the first assessment template; and
a report generation component comprising a report generation tool and a report generation utility library stored in the memory wherein, when executed by the at least one processor;
the report generation tool reads the first completed patient assessment entry from the assessment table from the data store,
the report generation tool provides the first completed assessment entry to the report generation utility library,
the report generation utility library creates a first plurality of Java objects comprising the values of the first patient assessment based on the first assessment template,
the report generation utility library provides the first plurality of Java objects to the report generation tool,
the report generation tool generates a first assessment report based on the first plurality of Java objects, wherein the first assessment report presents the values of the first patient assessment in a first format based on the first assessment template,
the report generation tool reads the second completed patient assessment entry from the assessment table from the data store,
the report generation tool provides the second completed assessment entry to the report generation utility library,
the report generation utility library creates a second plurality of Java objects comprising the values of the second patient assessment based on the second assessment template,
the report generation utility library provides the second plurality of Java objects to the report generation tool, and
the report generation tool generates a second assessment report based on the second plurality of Java objects, wherein the second assessment report presents the values of the second patient assessment in a second format based on the second assessment template.

11. The system of claim 10, wherein the system is a cloud computing system.

12. The system of claim 11, wherein the assessment component is executed by the at least one processor in a multi-tenant mode of operation, serving a plurality of different hospice care organizations.

13. The system of claim 10, wherein the data store is a PostgreSQL data store.

14. The system of claim 10, wherein the first and second assessment report each are formatted in portable document format (PDF) form.

15. A method of dynamically generating a patient assessment report automatically adapted to an assessment format used in conducting a patient assessment, comprising:
storing an assessment template in a format comprising a plurality of human readable attribute-value pairs in a column of a table in a data store, wherein the assessment template defines a presentation of a patient assessment form by a patient care giver tool UI as a plurality of sections of a patient assessment;
receiving values of a completed section of a patient assessment from the patient care giver tool UI by an assessment component executing on a computer system;
storing the values of the completed section of the patient assessment along with an associated section portion of the assessment template by the assessment component in an entry associated with the completed section of the patient assessment in an assessment section data table of the data store;
reading a plurality of entries associated with the completed sections of the patient assessment by the assessment component from the assessment section data table;
storing a completed patient assessment entry in an assessment table of the data store by the assessment component, wherein the entry comprises the values of the completed sections of the patient assessment and the assessment template;
reading the completed patient assessment entry from the assessment table from the data store by a report generation component executing on the computer system, where the report generation component comprises a report generation tool and a report generation utility library;
providing the values of the completed sections of the patient assessment and providing the assessment template by the report generation tool to the report generation utility library;
creating a plurality of Java objects comprising the values of the completed sections of the patient assessment by the report generation utility library based on the assessment template;
providing the plurality of Java objects by the report generation utility library to the report generation tool; and
generating a assessment report by the report generation tool based on the plurality of Java objects, wherein the assessment report presents the values of the patient assessment in a format based on the assessment template.

16. The method of claim 15, wherein the report generation utility library comprises an identity-to-name mapping JSON that defines associations of section identities to section text names and associations of card identities to card text names, wherein the report generation utility library comprises a plurality of card-type specific methods, wherein creating the first plurality of Java objects and the second plurality of Java objects comprises the report generation tool invoking a plurality of the card-type specific methods and the invoked card-type specific methods creating the Java objects, and wherein creating the plurality of Java objects is based at least in part on the associations of section identities to section text names and on the associations of card identities to card text names.

17. The method of claim 15, further comprising reading associations of section identities to section text names and reading associations of card identities to card text names from the data store by the report generation component, wherein the report generation utility library comprises a plurality of card-type specific methods, wherein creating the first plurality of Java objects and the second plurality of Java objects comprises the report generation tool invoking a plurality of the card-type specific methods and the invoked card-type specific methods creating the Java objects, and wherein creating the plurality of Java objects is based at least in part on the associations of section identities to section text names and on the associations of card identities to card text names.

18. The method of claim 15, wherein the report generation tool comprises Jasper report generation software.

19. The method of claim 15, wherein the report generation utility library is provided as a Java archive (JAR) file.

20. The method of claim 15, wherein the Java objects are Java lists or are Java beans.
